(19) United States
(12) Patent Application Publication
(10) Pub. No.: US 2024/0383786 A1
(43) Pub. Date: Nov. 21, 2024

(54) GRANULATION-PROMOTING MICROCARRIER FOR ANAEROBIC AMMONIUM OXIDATION (ANAMMOX) PROCESS, AND PREPARATION AND USE METHOD THEREOF

(71) Applicant: TONGJI UNIVERSITY, Shanghai (CN)

(72) Inventors: Xiaoli CHAI, Shanghai (CN); Pengcheng WANG, Shanghai (CN); Bin LU, Shanghai (CN)

(73) Assignee: TONGJI UNIVERSITY, Shanghai (CN)

(21) Appl. No.: 18/412,918

(22) Filed: Jan. 15, 2024

(30) Foreign Application Priority Data
May 16, 2023 (CN) 202310549282.X

(51) Int. Cl. C02F 3/28; C02F 3/00; C02F 3/30; C02F 101/16; C12M 1/12

(52) U.S. Cl. CPC C02F 3/2806; C02F 3/307; C12M 25/16; C02F 2003/001; C02F 2101/16; C02F 2305/06; C02F 2305/14

(57) ABSTRACT

Provided are a granulation-promoting microcarrier for an anaerobic ammonium oxidation (Anammox) process, and a preparation and use method thereof. The granulation-promoting microcarrier for the Anammox process is prepared by mixing a functional component, a regulatory component, and a structural component; wherein the functional component is an iron-based material; the regulatory component is a phase-change material; and the structural component includes a framework material and a foaming agent.

### CROSS REFERENCE TO RELATED APPLICATION

This patent application claims the benefit and priority of Chinese Patent Application No. 202310549282.X filed with the China National Intellectual Property Administration on May 16, 2023, the disclosure of which is incorporated by reference herein in its entirety as part of the present application.

### TECHNICAL FIELD

The present disclosure relates to the technical field of wastewater treatment, and in particular to a granulation-promoting microcarrier for an anaerobic ammonium oxidation (Anammox) process, and a preparation and use method thereof.

### BACKGROUND

Anaerobic ammonium oxidation (Anammox) process has received increasing attention due to an ability in achieving autotrophic and efficient nitrogen removal of wastewater. However, in traditional reactors, anaerobic ammonium oxidation bacteria (AnAOB) are easily lost, resulting in slower process start-up and poor stability, which are mostly solved by the granular sludge technology or biofilm technology at present. Anammox granular sludge technology has been widely studied and used due to higher biomass and nitrogen removal load, but its granulation speed and stability still to be improved.

### SUMMARY

In order to solve the deficiencies and defects of Anammox process, such as slow granulation and weak operation stability, the present disclosure is intended to provide a granulation-promoting microcarrier for an Anammox process, and a preparation and use method thereof. In the present disclosure, the granulation-promoting microcarrier for an Anammox process has the advantages of fast process start-up, strong stability, low cost, simple preparation process, wide application range, and desirable hydraulic performances, etc.

The objects of the present disclosure can be achieved by the following technical solutions:

A first object of the present disclosure is to provide a granulation-promoting microcarrier for an Anammox process, including a functional component, a regulatory component, and a structural component, wherein
the functional component is a pretreated iron-based material;
the regulatory component is a phase-change material;
the structural component is a mixture of a framework material and a foaming agent;
a mass ratio of the functional component, the regulatory component, and the structural component is in a range of (3-7):(1-3):(1-5).

In an embodiment of the present disclosure, the iron-based material is selected from the group consisting of an iron-based metal organic framework (Fe-MOF) and ferrous carbonate; and the iron-based material is used to release iron and ferrous ions so as to stimulate activity of AnAOB and promote aggregation and granulation of the AnAOB.

In an embodiment of the present disclosure, the phase-change material is selected from the group consisting of a 35° C. phase-change microcapsule, a phase-change silica gel, and a phase-change fiber; and the phase-change material is used to buffer changes in a wastewater temperature during long-term operation, thereby reducing impact of temperature changes on the AnAOB, and enhancing operation stability of the Anammox granular sludge process.

In an embodiment of the present disclosure, the framework material is selected from the group consisting of polylactic acid (PLA) and polyvinyl alcohol (PVA); and the framework material is used to bond various components, thereby controlling release speed of the functional component, and extending a service life of the carrier.

In an embodiment of the present disclosure, the foaming agent is selected from the group consisting of sodium lauryl sulfate (SLS) and sodium alcohol ether sulphate (AES); and the foaming agent is used to increase porosity of the material.

In an embodiment of the present disclosure, the pretreatment is performed by crushing and then sieving; and

during the sieving, a sieve has a mesh size of 500 mesh to 1,000 mesh.

In an embodiment of the present disclosure, a dosage ratio of the framework material to the foaming agent is in a range of (2-6):1.

In an embodiment of the present disclosure, the phase-change material has a particle size of 5 μm to 10 μm.

The second object of the present disclosure is to provide a method for preparation the granulation-promoting microcarrier for the Anammox process, including the following steps:

mixing evenly the functional component, the regulatory component, and the structural component to obtain a mixture, and subjecting the mixture to a post-treatment to obtain a granulation-promoting microcarrier with a particle size of 100 μm to 600 μm.

In an embodiment of the present disclosure, the post-treatment includes pyrolytic melting, mechanical foaming, cooling shaping, and then prilling.

In an embodiment of the present disclosure, the pyrolytic melting is conducted at a temperature of 155° C. to 170° C.;
the mechanical foaming is conducted at a stirring speed of 100 rpm to 300 rpm;
the cooling shaping is natural shaping at room temperature under ventilation; and
the prilling is conducted by mechanical crushing and then sieving.

The third object of the present disclosure is to provide use of the granulation-promoting microcarrier for the Anammox process in an Anammox system, wherein the granulation-promoting microcarrier and an inoculated sludge are added together into the Anammox system to treat wastewater.

In an embodiment of the present disclosure, the granulation-promoting microcarrier is added in an amount of 1 g/L to 3 g/L.

In an embodiment of the present disclosure, the granulation-promoting microcarrier has a particle size of 100 μm to 600 μm.

In the present disclosure, adding inert micro-nuclei into the Anammox system could promote the granulation of Anammox sludge; meanwhile, iron could stimulate the growth and metabolism of AnAOB, thereby playing a key role in the nitrogen removal and growth of AnAOB.

Comparing with the prior art, some embodiments of the present disclosure have the following beneficial effects:
(1) In the present disclosure, a stimulation strategy is adopted that the iron-based material is used to strengthen the activity of AnAOB, thereby improving the formation speed, nitrogen removal efficiency, and stability of Anammox granular sludge.
(2) In the present disclosure, the phase-change material strengthens temperature buffering performance of the granulation-promoting microcarrier, and reduces the impact of changes in the wastewater temperature on the AnAOB attached to a surface of the granulation-promoting microcarrier, thereby enhancing the stability of the Anammox granular sludge.
(3) In the present disclosure, a process of shaping and then crushing is adopted, such that a size of the finished product could be customized according to the usage scenario. By appropriately increasing the size of the microcarrier, the application range of the microcarrier could be expanded to biofilm processes such as biofilters. The obtained microcarrier has the advantages of low cost, simple preparation process, wide application range, and desirable hydraulic performance, etc.

### BRIEF DESCRIPTION OF THE DRAWINGS

FIG. 1 shows a schematic flow chart of a method for preparing a granulation-promoting microcarrier for an Anammox process according to an embodiment of the present disclosure; and

FIG. 2 shows a schematic diagram of use of the granulation-promoting microcarrier in an anaerobic sludge bed according to an embodiment of the present disclosure.

### REFERENCE NUMERALS

1 refers to a granulation-promoting microcarrier; 2. refers to an influent; 3. refers to an effluent flume; and 4. refers to an effluent.

### DETAILED DESCRIPTION OF THE EMBODIMENTS

The present disclosure provides a granulation-promoting microcarrier for an Anammox process, including a functional component, a regulatory component, and a structural component, wherein
the functional component is a pretreated iron-based material;
the regulatory component is a phase-change material; and
the structural component is a mixture of a framework material and a foaming agent;
a mass ratio of the functional component, the regulatory component, and the structural component is in a range of (3-7):(1-3):(1-5).

In an embodiment of the present disclosure, the iron-based material is selected from the group consisting of an iron-based metal organic framework (Fe-MOF) and ferrous carbonate; and the iron-based material is used to release iron and ferrous ions so as to stimulate activity of AnAOB and promote aggregation and granulation of the AnAOB.

In an embodiment of the present disclosure, the phase-change material is selected from the group consisting of a 35° C. phase-change microcapsule, a phase-change silica gel, and a phase-change fiber; and the phase-change material is used to buffer changes in a wastewater temperature during long-term operation, thereby reducing impact of temperature changes on the AnAOB, and enhancing operational stability of the Anammox granular sludge process.

In an embodiment of the present disclosure, the framework material is selected from the group consisting of polylactic acid (PLA) and polyvinyl alcohol (PVA); and the framework material is used to bond various components, thereby controlling release speed of the functional component, and extending a service life of the carrier.

In an embodiment of the present disclosure, the foaming agent is selected from the group consisting of sodium lauryl sulfate (SLS) and sodium alcohol ether sulphate (AES); and the foaming agent is used to increase porosity of the material.

In an embodiment of the present disclosure, the pretreatment is performed by crushing and then sieving; and
during the sieving, a sieve has a mesh size of 500 mesh to 1,000 mesh.

In an embodiment of the present disclosure, a dosage ratio of the framework material to the foaming agent is in a range of (2-6): 1.

In an embodiment of the present disclosure, the phase-change material has a particle size of m to 10 μm.

The present disclosure provides a method for preparation the granulation-promoting microcarrier for the Anammox process, including the following steps:
mixing evenly the functional component, the regulatory component, and the structural component to obtain a mixture, and subjecting the mixture to a post-treatment to obtain a granulation-promoting microcarrier with a particle size of 100 μm to 600 μm.

In an embodiment of the present disclosure, the post-treatment includes pyrolytic melting, mechanical foaming, cooling shaping, and then prilling.

In an embodiment of the present disclosure, the pyrolytic melting is conducted at a temperature of 155° C. to 170° C.;
the mechanical foaming is conducted at a stirring speed of 100 rpm to 300 rpm;
the cooling shaping is natural shaping at room temperature under ventilation; and
the prilling is conducted by mechanical crushing and then sieving.

The present disclosure provides use of the granulation-promoting microcarrier for the Anammox process in an Anammox system, wherein the granulation-promoting microcarrier and an inoculated sludge are added together into the Anammox system to treat wastewater.

In an embodiment of the present disclosure, the granulation-promoting microcarrier is added in an amount of 1 g/L to 3 g/L.

In an embodiment of the present disclosure, the granulation-promoting microcarrier has a particle size of 100 μm to 600 μm.

The present disclosure will be described in detail below with reference to the drawings and specific embodiments.

In the following examples, unless otherwise specified, the reagents used are all commercially available reagents; the detection means and methods used are all conventional detection means and methods in this field.

### Example 1

This example provided a granulation-promoting microcarrier for an Anammox process and a preparation method thereof.

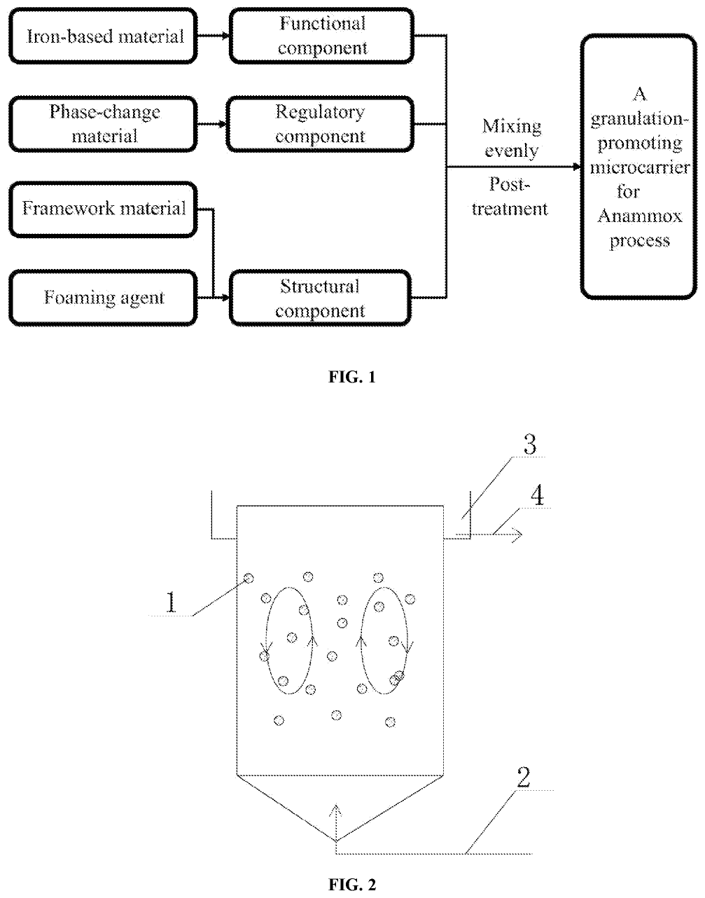

The preparation method was performed by the following steps (as shown in FIG. 1):
(S1) Ferrous carbonate was crushed and sieved through a 1,000 mesh sieve to obtain a functional component; a 35° C. phase-change microcapsule with a particle size of 5 μm was used as an regulatory component; and PLA and SLS were mixed evenly at a mass ratio of 5:1 to obtain a structural component.
(S2) The functional component, the regulatory component, and the structural component obtained in step (S1) were mixed evenly at a mass ratio of 5:2:3 to obtain a mixture; the mixture was subjected to pyrolytic melting at 170° C., mechanical foaming (at a stirring speed of 250 rpm to 300 rpm), cooling shaping at room temperature under ventilation, mechanical crushing, and sieving in sequence to obtain a particle with a particle size of 300 μm to 400 μm, i.e. the granulation-promoting microcarrier.

### Example 2

This example provided use of the granulation-promoting microcarrier for an Anammox process obtained in Example 1 in an up-flow anaerobic sludge bed.

The granulation-promoting microcarrier prepared in Example 1 and an inoculated sludge were added together into the up-flow anaerobic sludge bed (an initial particle size of the inoculated sludge was 2 μm to 200 μm), and a dosage of the granulation-promoting microcarrier prepared in Example 1 was 1 g/L (based on a volume of the anaerobic sludge bed). As shown in FIG. 2, influent 2 was introduced from a bottom of the anaerobic sludge bed, the granulation-promoting microcarrier 1 was located inside the anaerobic sludge bed, and effluent 4 was discharged through an effluent flume 3 of the anaerobic sludge bed. During operation, a nitrogen removal rate of the up-flow anaerobic sludge bed could reach not less than 80% stably, and a particle size of a mature Anammox granular sludge formed in the anaerobic sludge bed is in a range of 900 μm to 2,600 μm.

The above description of the embodiments is for the convenience of a person of ordinary skill in the technical field to understand and use the present disclosure. It is obvious that those skilled in the art could easily make various modifications to these embodiments and apply the general principles described herein to other embodiments without creative efforts. Therefore, the present disclosure is not limited to the above-mentioned embodiments, and the improvements and modifications made by those skilled in the art without departing from the scope of the present disclosure should be within the scope of the present disclosure.

1. A granulation-promoting microcarrier for an anaerobic ammonium oxidation (Anammox) process, comprising a functional component, a regulatory component, and a structural component, wherein
the functional component is an iron-based material;
the regulatory component is a phase-change material;
the structural component is a mixture of a framework material and a foaming agent, a mass ratio of the framework material to the foaming agent is in a range of (2-6):1; and
a mass ratio of the functional component, the regulatory component, and the structural component is in a range of (3-7):(1-3):(1-5).

2. The granulation-promoting microcarrier for the Anammox process of claim 1, wherein the iron-based material is selected from the group consisting of an iron-based metal organic framework (Fe-MOF) and ferrous carbonate.

3. The granulation-promoting microcarrier for the Anammox process of claim 1, wherein the phase-change material is selected from the group consisting of a 35° C. phase-change microcapsule, a phase-change silica gel, and a phase-change fiber.

4. The granulation-promoting microcarrier for the Anammox process of claim 1, wherein the phase-change material has a particle size of 5 μm to 10 μm.

5. The granulation-promoting microcarrier for the Anammox process of claim 1, wherein the framework material is selected from the group consisting of polylactic acid (PLA) and polyvinyl alcohol (PVA); and
the foaming agent is selected from the group consisting of sodium lauryl sulfate (SLS) and sodium alcohol ether sulphate (AES).

6. A method for preparing the granulation-promoting microcarrier for the Anammox process of claim 1, comprising the following steps:
mixing evenly the functional component, the regulatory component, and the structural component to obtain a mixture, and subjecting the mixture to a post-treatment to obtain a granulation-promoting microcarrier with a particle size of 100 μm to 600 μm.

7. The method of claim 6, wherein the post-treatment comprises pyrolytic melting, mechanical foaming, cooling shaping, and then prilling.

8. The method of claim 7, wherein the pyrolytic melting is conducted at a temperature of 155° C. to 170° C.;
the mechanical foaming is conducted at a stirring speed of 100 rpm to 300 rpm;
the cooling shaping is natural shaping at room temperature under ventilation; and
the prilling is conducted by mechanical crushing and then sieving.

9. A method for utilizing the granulation-promoting microcarrier for the Anammox process of claim 1 in an Anammox system, comprising adding the granulation-promoting microcarrier and an inoculated sludge into the Anammox system to treat wastewater.

10. The method of claim 9, wherein the granulation-promoting microcarrier is added in an amount of 1 g/L to 3 g/L.

11. The method of claim 6, wherein the iron-based material is selected from the group consisting of an iron-based metal organic framework (Fe-MOF) and ferrous carbonate.

12. The method of claim 6, wherein the phase-change material is selected from the group consisting of a 35° C. phase-change microcapsule, a phase-change silica gel, and a phase-change fiber.

13. The method of claim 6, wherein the phase-change material has a particle size of 5 μm to 10 μm.

14. The method of claim 6, wherein the framework material is selected from the group consisting of polylactic acid (PLA) and polyvinyl alcohol (PVA); and
the foaming agent is selected from the group consisting of sodium lauryl sulfate (SLS) and sodium alcohol ether sulphate (AES).

15. The method of claim 9, wherein the iron-based material is selected from the group consisting of an iron-based metal organic framework (Fe-MOF) and ferrous carbonate.

16. The method of claim 9, wherein the phase-change material is selected from the group consisting of a 35° C. phase-change microcapsule, a phase-change silica gel, and a phase-change fiber.

17. The method of claim 9, wherein the phase-change material has a particle size of 5 μm to 10 μm.

18. The method of claim 9, wherein the framework material is selected from the group consisting of polylactic acid (PLA) and polyvinyl alcohol (PVA); and
the foaming agent is selected from the group consisting of sodium lauryl sulfate (SLS) and sodium alcohol ether sulphate (AES).
